(19) United States
(12) Patent Application Publication
(10) Pub. No.: US 2016/0302786 A1
(43) Pub. Date: Oct. 20, 2016

(54) Apparatus And Method for Securing Flexible Member

(71) Applicant: ZIPTEK LLC, Sarasota, FL (US)

(72) Inventors: William F. Bennett, Sarasota, FL (US); Ramses Galaz Mendez, Hermosillo (MX); Daniel Francisco Gomez Romo, Hermosillo (MX)

(21) Appl. No.: 15/099,932

(22) Filed: Apr. 15, 2016

Related U.S. Application Data
(60) Provisional application No. 62/258,908, filed on Nov. 23, 2015.
(60) Provisional application No. 62/149,412, filed on Apr. 17, 2015.

(51) Int. Cl. A61B 17/04

(52) U.S. Cl. CPC A61B 17/0401; A61B 2017/0451; A61B 2017/0414; A61B 2017/0454; A61B 2017/0409

(57) ABSTRACT

An apparatus and method is disclosed for securing tissue using an improved retention device. The apparatus and method is suitable for securing a suture to a bone or an anchor or securing a first tissue to a second tissue. The retention device is capable of attaching to a flexible member. The apparatus and method is appropriate for arthroscopic surgery.

### CROSS-REFERENCE TO RELATED APPLICATIONS

This application claims benefit of U.S. Patent Provisional application No. 62/149,412 filed 17 Apr. 2015 and claims benefit of U.S. Patent Provisional application No. 62/258,908 filed 23 Nov. 2015. All subject matter set forth in provisional application No. 62/149,412 and 62/258,908 is hereby incorporated by reference into the present application as if fully set forth herein.

### BACKGROUND OF THE INVENTION

1. Field of the Invention

This invention relates to surgery and more particularly to an apparatus and method for securing tissue with a suture. Furthermore, the invention relates to an improved retention device capable of attaching to a flexible member.

2. Description of the Related Art

In recent years, suture retention devices or captures have found increased use in the surgical art. Typically, a capture is used in association with a suture for securing an end of the suture without the need for tying a knot in the suture. The use of a capture has several advantages over the traditional tying of a suture. Firstly, a capture will secure a suture faster than tying a traditional knot. Secondly, a capture will generally yield more consistent holding strength relative to a traditional knot. Thirdly, a capture can be used in arthroscopic surgery where space limitation makes knot tying difficult for a surgeon.

The following prior art are examples of the development of captures and the like, including anchors, fasteners and crews for the surgical art. These prior art examples may be used in various types of surgery including medical, veterinary and dental implants.

U.S. Pat. No. 4,898,156 to Gatturna, et al. discloses a suture anchor for anchoring one end of a conventional suture to bone comprising a coupling member, with at least one barb made of a resilient elastic material projecting from the coupling member, and structure for use in attaching one end of a suture to the suture anchor. The coupling member and the at least one barb are adapted to securely anchor one end of the suture in bone when the suture is attached to the attachment structure, so that the other end of the suture extending outside the bone can be used to attach objects to the bone. A novel application tool is also provided.

U.S. Pat. No. 5,733,307 to Dinsdale discloses a bone anchor with a suture trough for attaching bone to tissue using a suture. The bone anchor includes a threaded bone anchor body having a major diameter, a minor diameter, a tip, and a drive head. A suture trough is firmed within the anchor body with a width greater than or equal to the diameter of the suture and a depth below the minor diameter greater than or equal to the diameter of the suture. A suture passageway connects the opposing portions of the suture trough. The suture seats within the suture trough during insertion of the bone anchor into the bone.

U.S. Pat. No. 5,370,661 to Branch discloses a method for attaching tissue to a bone, which includes the use of as filament member and a head member which engages the filament member and holds a tissue in place. Also provided is a device and method for repairing a break in tissue, which indicates the use of a filament member and at least one member which engages the filament member.

U.S. Pat. No. 5,413,585 to Pagedas discloses a self locking suture lock having a first suture thread opening in which the thread is secured before the surgical stitch and a cone shaped second stitch lock opening that is larger at the front side than at the back side, and designed to receive suture thread in only one direction, thereby locking it against withdrawal after the stitch to complete and lock it without the need for a surgical knot. A tongue in the second opening will allow passage through the cone shaped second opening from front side to back side but not allow passage from back to side to front side or pulling out of second opening once threaded. The front side of the self locking suture may be distinctively colored so that a surgeon will know which side of the suture lock will accept the suture thread. The first opening may take the form of a deformable slot, a pair of openings, or other forms. The tongue may engage the edge of the second opening, a slot, or other unidirectional lock structures.

U.S. Pat. No. 5,584,835 to Greenfield discloses a two-part device for suturing soli tissue to bone. The device employs a bone anchor which is installed in the bone and a suture anchor which is coupled to the soft tissue and then engaged with the bone anchor. The engagement of the suture anchor with the bone anchor is readily reversible so as to permit adjustments in the placement of the sutures. In the practice of the invention, no step is irreversible except the drilling of the bone hole that accepts the bone anchor. A special tool is described for facilitating the installation of a bone anchor having a round internal cross-section. The suture anchor can be installed with the application of only direct pressure, and can be disengaged by counterclockwise rotation, with the use of a disengagement tool, such as a screw driver. Sutures are threaded through apertures in the suture anchor, and the engagement of the sutures is enhanced by a friction fit between the suture and bone anchors.

U.S. Pat. No. 5,938,686 to Benderev, et al. discloses a bone anchor implantation device positioned over a bone, and a bone anchor installed in a bone.

U.S. Pat. No. 5,948,000 to Larsen, et al. discloses a system for suture anchor placement including an apparatus having a handle portion and an operating portion. The handle portion includes a spring, a needle park, and a member for releasably holding a length of the suture. The operating portion includes a sheath tube and a plunger rod slidably disposed within the bore of the sheath tube. The plunger rod is fixedly mounted at its proximal end to the handle. The suture anchor is releasably engaged to the distal end portion of the plunger rod. The sheath tube is mounted to the handle and movable with respect to the handle between a distal position and a proximal position, the sheath tube being resiliently biased to the distal position by the spring and movable to the proximal position in response to proximally directed force of sufficient magnitude applied to the distal end of the sheath tube. The sheath tube has a portion with an outer diameter greater than the diameter of the hole in the bone such that when the installation tool is pressed toward the bone, the sheath tube retracts into the handle and the suture anchor is advanced into a hole previously made in the bone. The suture, initially held in a taut configuration, is released in response to movement of the sheath tube to its proximal position.

U.S. Pat. No. 6,013,083 to Bennett discloses torn tissue such as a rotator cuff positioned on the bone exterior by a tissue grasper. A cannula is inserted through the skin substantially to the torn tissue. A drill guide is inserted into the cannula, a drill bit is inserted into the drill guide, and a hole is drilled through the torn tissue and completely through the bone. The drill bit is removed and an inner cannula is passed through the drill guide until its distal end is engaged on the tom tissue or alternatively passed through the hole until its distal end is at the far end of the drilled hole. A soft tissue anchor having expandable wings at its distal end and sutures secured to an eyelet at its proximal end is releasably connected to the distal end of a tubular deployment tool with the free ends of the sutures extending through the deployment tool. The deployment tool is passed through the inner cannula and drilled hole until the expandable wings clear the far end of the hole a sufficient distance to allow the wings to expand to a diameter larger than the diameter of the drilled hole. The deployment tool, inner cannula, drill guide, and cannula are removed and tension is applied to the suture to engage the expanded wings of the anchor on the exterior surface of the bone surrounding the drilled hole. A button is run down on the sutures through the cannula and secured on the torn tissue by the sutures such that the torn tissue is secured to the bone and the sutures are anchored to the hard exterior surface of the bone by the expanded anchor.

U.S. Pat. No. 6,015,410 to Tormala, et al. discloses a bioabsorbable surgical implant for use in supporting soft tissue in a superior position in the body. The surgical implant includes a shaft that connects the implant to a bone or hard tissue and a head secured to the shaft. The head of the implant has a concave lower surface so that a suture (or sutures) can easily be wound around the shaft below the head and locked to this position by tightening the head against bone or hard tissue and by pushing or turning the shaft into the bone or hard tissue. The connected shaft and head are formed from a resorbable polymer, copolymer, polymer alloy or composite, which maintains a specified strength for a period of time at least equal to a healing period for the patient. Thereafter, the surgical implant is substantially reabsorbed by the body over a period of time needed for healing. The surgical implant is particularly adapted for use in endoscopic face and/or brow lift surgery and other endoscopic cosmetic, plastic and reconstructive surgical procedures, where sutures are applied for tissue lifting.

U.S. Pat. No. 6,117,162 to Schmieding, et al. discloses a corkscrew suture anchor having a continuous thread spiralling around a tapering central core. At the distal end, the suture anchor terminates in a rounded point. At the proximal end of the suture anchor is an eye for receiving suture. The suture anchor has a large thread surface per turn of thread. Anti-backout ridges can be formed on the front and/or back faces of the threads. A driver liar the suture anchor is provided, the driver including a shaft having a central axis, a length, a distal end, and a proximal end. The shaft is provided at its distal end with an opening aligned with the central axis of the shaft, for receiving the hexagonal proximal end of the suture anchor. One or more sutures threaded through the suture eye are threaded through the hollow tubular shaft. The suture is pulled into and captured by V-shaped notches on the proximal end of the handle to hold the suture anchor in place on the distal end of the driver under the tension of the captured sutures.

U.S. Pat. No. 6,206,886 to Bennett discloses torn tissue such as a rotator cuff positioned on the bone exterior by a tissue grasper. A cannula is inserted through the skin substantially to the torn tissue. A drill guide is inserted into the cannula, a drill bit is inserted into the drill guide, and a hole is drilled through the torn tissue and completely through the bone. The drill bit is removed and an inner cannula is passed through the drill guide until its distal end is engaged on the torn tissue or alternatively passed through the hole until its distal end is at the far end of the chilled hole. A soft tissue anchor having expandable wings at its distal end and sutures secured to an eyelet at its proximal end is releasably connected to the distal end of a tubular deployment tool with the free ends of the sutures extending through the deployment tool. The deployment tool is passed through the inner cannula and drilled hole until the expandable wings clear the far end of the hole a sufficient distance to allow the wings to expand to a diameter larger than the diameter of the drilled hole. The deployment tool, inner cannula, drill guide, and cannula are removed and tension is applied to the suture to engage the expanded wings of the anchor on the exterior surface of the bone surrounding the drilled hole. A button is run down on the sutures through the cannula and secured on the torn tissue by the sutures such that the torn tissue is secured to the bone and the sutures are anchored to the hard exterior surface of the bone by the expanded anchor.

U.S. Pat. No. 6,214,031 to Schmieding, et al. discloses a corkscrew suture anchor having a continuous thread spiraling around a tapering central core. At the distal end, the suture anchor terminates in a rounded point. At the proximal end at the suture anchor is an eye for receiving suture. The suture anchor has a large thread surface per turn of thread. Anti-backout ridges can be formed on the front and/or back faces of the threads. A driver for the suture anchor is provided, the driver including a shaft having a central axis, a length, a distal end, and a proximal end. The shaft is provided at its distal end with an opening aligned with the central axis of the shaft, for receiving the hexagonal proximal end of the suture anchor. One or more sutures threaded through the suture eye are threaded through the hollow tubular shaft. The suture is pulled into and captured by V-shaped notches on the proximal end of the handle to hold the suture anchor in place on the distal end of the driver under the tension of the captured sutures.

U.S. Pat. No. 6,293,961 to Schwartz, et al. discloses a device for locking a suture in place without the need for tying knots once the suture is placed within tissue. The device comprises an anchor having a front section and a rear section, a cannula extending through the front and rear sections through which the suture extends, and a head positioned adjacent an end of the suture for locking the suture within the cannula.

U.S. Pat. No. 6,491,714 to Bennett discloses a surgical apparatus for anchoring and reattachment of torn tissue such as a rotator cuff against on the outer or exterior surface of a tissue substrate. The invention includes a tissue substrate anchor such as that having expandable wings, an elongated suture member securable at its proximal end to the tissue substrate anchor, and a torn tissue retainer lockingly attachable along the length of the suture member by mating interlocking structure therebetween. Tension is applied to the free distal end of the suture member while the tissue retainer is non-reversibly moved longitudinally along on the suture member to secure the torn tissue against the tissue substrate outer surface. A separate torn tissue gripping member may also be provided for broadened and enhanced torn tissue securement against the tissue substrate. In another embodiment, the tissue anchor is formed as a unit with the suture member enabling additional forms of tissue repair such as the closure of an internal meniscus tear or separation.

U.S. Pat. No. 6,533,802 to Bojarski, et al. discloses a method of securing a tissue graft within a bone passage including providing a graft fixation member comprising a closed-loop having a pair of opposing loop sections and capturing a first loop section of the closed-loop within the fixation member. An opposing second loop section of the closed loop is passed through an opening in the tissue graft, and the second loop section of the closed loop is secured to the fixation member.

U.S. Pat. No. 7,303,577 to Dean discloses a method, system and apparatus for augmenting the surgical repair of soft tissue injuries, in which a first end of a bridge member attaches to a first portion Of healthy tissue, and a second end a the bridge member attaches to a second portion of healthy tissue. The bridge member (or bridge members) used to augment the soft tissue repair may be interconnected or function independently. Flexibility and elasticity of the bridge member are determined by the situation and may be altered to improve healing. The device may he used in arthroscopic procedures, and may be manufactured in a variety of lengths, or may be manufactured one length and be cut to the desired length, or otherwise altered to provide an optimal length of the bridge member.

U.S. Pat. No. 7,530,990 to Perriello, et al. discloses a method of securing a tissue graft within a bone passage including providing a graft fixation member comprising a closed double-loop having a pair of differently sized loop sections and capturing both sections of the closed-loop within the fixation member. The longer loop section is passed through an opening in the tissue graft then is passed through the smaller loop and thrown over the fixation member to form a knot.

U.S. Pat. No. 7,585311 to Green, et al. discloses a method and device for securing soft tissue to a rigid material such as bone. A bone anchor is described that comprises a base and a top such that suture material may be compressed between surfaces on the base and top to secure the suture to the anchor. Also described is an inserter that can be used to insert the bone anchor into bone and move the anchor top relative to the anchor base to clamp suture material there between. Also described is a soft-tissue and bone piercing anchor and associated inserter. Methods are described that allow use of the bone anchors to provide multiple lengths of suture material to compress a large area of soft tissue against bone.

U.S. Pat. No. 7,615,061 to White, et al. discloses a suture-loading system, method and apparatus for loading, a suture onto a bone anchor. The system comprising: a bone anchor comprising a suture leg-anchoring structure and a plurality of body holes on the anchor; a suture comprising a standing end portion and a working end portion; a standing end trackway to guide the standing end portion of the suture through the suture leg-anchoring structure; and a working end trackway to guide the working end portion of the suture through the body holes in the anchor.

U.S. Pat. No. 7,637,926 to Foerster, et al. discloses an innovative bone anchor and method for securing soft tissue, such as tendons, to bone, which permit a suture attachment that lies entirely beneath the cortical bone surface. Advantageously, the suturing material between the soft tissue and the bone anchor is secured without the need for tying a knot. The suture attachment to the bone anchor involves the looping of a length of suture around a pulley within the bone anchor, tightening the suture and attached soft tissue, and clamping the suture within the bone anchor. The bone anchor may be a tubular body having a lumen containing a plurality of suture-locking elements that clamp the suture therein. The locking elements may be thin and C-shaped. One or more locking plugs attached to separable actuation rods displace axially within the lumen and act on the locking elements to displace them radially. A generally uniform passage through the locking elements in their first positions converts to a smaller irregular passage after the locking plug displaces the elements to their second positions, thus effectively clamping the suture. The bone anchor timber may include locking structure for securing itself within a bone cavity.

U.S. Pat. No. 7,658,750 to Li discloses a suture anchoring system and method including a plurality of anchor members interconnected to form an anchor assembly with a suture extending therefrom. The anchor assembly has an insertion configuration wherein the anchor members are aligned in a substantially linear arrangement for delivery through an aperture in bodily tissue and an expanded configuration wherein the anchor members are transitioned to a non-linear arrangement to prevent passage of the anchor assembly back through the aperture.

U.S. Pat. No. 7,658,751 to Stone, et al. discloses a suture construction an method for forming a suture construction. The construction utilizes a suture having an enlarged central body portion defining a longitudinal passage. First and second ends of the suture are passed through first and second apertures associated with the longitudinal passage to form a pair of loops. Portions of the suture lay parallel to each other within the suture. Application of tension onto the suture construction causes constriction of the longitudinal passage, thus preventing relative motions of the captured portions of the suture.

U.S. Pat. No. 7,662,157 to Ahmad discloses a bone anchor including a screw portion configured to penetrate a bone. The screw portion includes a retention thread extending at least part of a length of the screw portion and a tip at a first end of the screw portion. The bone anchor includes a protrusion adjacent a second end of the screw portion. The second end is opposite the first end of the screw portion. The protrusion comprises a plurality of external sides forming a shape and a rounded interior surface enclosing a protrusion recess. The rounded interior surface includes a recess thread configured to retain a component at least partially in the protrusion recess. The protrusion has a maximum width that is less than a maximum diameter of the screw portion such that a shoulder is formed where the protrusion meets the second end of the screw portion.

U.S. Pat. No. 7,674,274 to Forester, et al. discloses a bone anchor device for attaching connective tissue to bone comprising a disk adapted for insertion into a portion of bone to which the connective tissue is to be attached. The disk is movable between a bent orientation for presenting a smaller cross-section and an expanded orientation for presenting a larger cross-section. The bent orientation is utilized for inserting the disk through a small hole into a region of cancellous bone beneath the cortical bone layer, after which the disk is actuated to its expanded orientation so that it will be permanently anchored in the cancellous bone, as it will be too large to return proximally through the hole in the cortical bone layer. Two embodiments are disclosed. In a first embodiment, the disk is initially formed in the expanded orientation, of spring steel. In a second embodiment, the disk is initially formed in the bent orientation, and spring steel is not required.

U.S. Pat. No. 7,686,838 to Wolf, et al. discloses a surgical anchor device for the repair of a torn ligament or tendon, primarily the anterior cruciate ligament in the knee. The device is used to affix the ligament within a femoral bone tunnel in the distal portion of the femur from the intra-articular surface. The device provides a pulley for a suture, wherein a free end of the suture may be pulled away from the device to draw the suture attached to the ligament graft within the femoral bone tunnel securing the ligament graft within the bone tunnel. Installation of the device is provided by insertion of the device through a tibial hole, through the femoral tunnel out of the lateral femoral cortex, pulling the attached sutures simultaneously to flatten the device against the lateral femoral cortex, attaching one end of the suture to the ligament graft and pulling the other end of the suture until the graft is situated properly within the femoral bone tunnel and tying the free end of the suture to retain the graft within the femoral bone tunnel.

U.S. Pat. No. 7,695,494 to Foerster discloses a device for attaching connective tissue to bone. The device has a longitudinal axis and comprises an annular toggle member and a body member disposed distally of the toggle member, such that there is an axial space between the toggle member and the body member. The toggle member is movable between an undeployed position wherein the toggle member has a smaller profile in a direction transverse to the axis and a deployed position wherein the toggle member has a larger profile in the direction transverse to the axis. When installed in a desired procedural site, in suitable bone, suturing material extends axially through a center aperture in the annular toggle member, without being secured to or contacting the toggle member. This approach permits a suture attachment which lies entirely beneath the cortical bone surface, and which further permit the attachment of suture to the bone anchor without the necessity for tying, knots, which is particularly arduous and technically demanding in the ease of arthroscopic procedures.

U.S. Pat. No. 7,713,285 to Stone, et al. discloses a suture anchor for anchoring a suture in the selected portion of an anatomical portion for fixing a suture thereto. The suture anchor includes an anatomical engaging portion and a suture engaging portion wherein both the anatomical engaging portion and a suture engaging portion are adapted to be substantially disposed below an exterior of the anatomical portion after implantation. Suture eyelets are provided in the suture engaging section and a suture passage is formed to interconnect the eyelets to allow a suture to be easily threaded in the suture engaging section from the first eyelet to the second eyelet.

United States Patent Application 2002/0004668 to Bartlett discloses a suture anchor comprising a generally quadrilaterally shaped body having a bore-abutting surface with a leading gripping edge at one end of the bore-abutting surface, a trailing gripping edge at the other end, and a closing surface having an arcuate portion and a linear portion engaging the leading and trailing gripping edges, respectively, generally opposite the bore-abutting surface. The body further defines a suture bore extending transversely therethrough and an inserter bore adapted to receive an insertion tool. A suture engages the suture anchor through the bore and is adapted to engage bodily tissue to he secured to the bone. Also provided are a knotless suture anchor configuration, an associated insertion tool, and a suture anchor kit, as well as a method of implanting a suture anchor in a bore in a bone.

United States Patent Application 2002/0004669 to Bartlett discloses a suture anchor comprising a generally quadrilaterally shaped body having a bore-abutting surface with a leading gripping edge at one end of the bore-abutting surface, a trailing gripping edge at the other end, and a closing surface having an arcuate portion and a linear portion engaging the leading and trailing gripping edges, respectively, generally opposite the bore-abutting surface. The body further defines a suture bore extending transversely therethrough and an inserter bore adapted to receive an insertion tool. A suture engages the suture anchor through the bore and is adapted to engage bodily tissue to be secured to the bone. Also provided are a knotless suture anchor configuration, an associated insertion tool, and a suture anchor kit, as well as a method of implanting a suture anchor in a bore in a bone.

United States patent Application 2002/0007196 to Bartlett discloses a suture anchor comprising a generally quadrilaterally shaped body having a bore-abutting surface with a leading gripping edge at one end of the bore-abutting surface, a trailing gripping edge at the other end, and a closing surface having an arcuate portion and a linear portion engaging the leading and trailing gripping edges, respectively, generally opposite the bore-abutting surface. The body further defines a suture bore extending transversely therethrough and an inserter bore adapted to receive an insertion tool. A suture engages the suture anchor through the bore and is adapted to engage bodily tissue to be secured to the bone. Also provided are a knotless suture anchor configuration, an associated insertion tool, and a suture anchor kit, as well as a method of implanting a suture anchor in a bore in a bone.

United States Patent Application 2006/0058844 to White, et al. discloses an internal tissue puncture closure method and apparatus providing a locking device for compressing and holding an external component such as a collagen sponge at a puncture situs. The locking device facilitates compression of the external component in a first direction, but prevents or locks against retraction.

United States Patent Application 2006/0106422 to Del Rio, et al. discloses suture tying in a medical procedure including the use of an anchor that includes a suture locking device. The device includes a sliding pin guided by axial side slots formed in the locking device that forces the suture where the two ends thereof are inserted into the interior of the suture locking mechanism to drive the captured suture and pin to fit into a recess on the top of the suture locking mechanism such that the top of the pin binds the suture against the upper inner surface of the recess. The recess can be serrated to enhance the locking capability and the pin is polygonal shaped to provide side surfaces that run parallel to the wall of the recess to assure that the side portion of the pin do not fracture the suture and cause it to weaken. The locking of the suture is automatic upon the deployment of the anchor which actuates the pin into the locking position, thus eliminating the necessity of manually knotting the suture and improving the cinching of the tissue to the bone. The suture locking mechanism is usable for attaching tissue to the bone, tying tissue to tissue and the like. The disclosure also includes the method of deploying the combined anchor and suture locking device in a medical procedure.

United States Patent Application 2007/015651 to Guan, et al. discloses an expanding plug for tendon fixation featuring a two-part system in which an expansion pin fits inside a fixation sleeve. The fixation sleeve is configured to expand diametrically to achieve interference fixation of a graft tendon inside of a bone tunnel. Fixation sleeve expansion is urged by a two-step engagement of the expansion pin. The tendon graft is assembled to the expanding bolt and situated within a bone tunnel. Passing suture is used to pull a joint-line end of the expansion pin into the tunnel to partially expand the fixation sleeve at the joint-line end. Pulling a graft end of the expansion pin toward the joint line expands the fixation sleeve to place the expanding plug, in the fully deployed configuration.

United States Patent Application 2007/028375 to Hindrichs, et al. discloses an implant structure for use in pulling two soli body tissue areas closer together in a patient (e.g., two points along or adjacent to the patient's mitral valve annulus) including at least two tissue anchor structures that are respectively implantable into the two tissue areas. A tether structure links the two tissue anchors and can be shortened and held in that condition by a cinch structure. Bracing structures are used between the anchors and the tether to help keep the longitudinal axes of the anchors transverse to the tether axis even when the tether is wider tension. The tether may he sheathed in a cushioning sleeve to help protect adjacent tissue from erosion by the tether.

United States Patent Application 2008/0082113 to Bishop et al. discloses embodiments of apparatus and methods for tissue lifting, or for correcting a ptosis condition caused by tissue stretching. In some embodiments a tissue is supported by a support member. In sonic embodiments, tension is applied to a support member through at least one suspension member. The described embodiments provide examples of methods and apparatus effective for use in lifting or otherwise applying tension to various tissues, including tissues of the breast, buttock, thigh, arm, abdomen, neck and face.

United States Patent Application 2008/0234731 to Leung, et al. discloses a suture anchor for approximating tissue to bone or other tissue. The suture anchor comprises an anchor member to fixedly engage the bone for securing the anchor member relative to the bone. A plurality of sutures are mounted to the proximal end of the anchor member so that the sutures extend outwardly from the anchor member. Each suture has a sharp pointed distal end for penetrating the tissue and a plurality of barbs extending from the periphery and disposed along the length of the body of the suture. The barbs permit movement of the sutures through the tissue in a direction of movement of the pointed end and prevent movement of the sutures relative to the tissue in a direction opposite the direction of movement of the pointed end. At least one pointed distal end of at least one suture comprises a needle.

United States Patent Application 2009/0248071 to Saint, et al. discloses methods and apparatus tot use in supporting tissue in a patient's body. In some embodiments, the patient's breast is supported. In some embodiments, the methods provide ways of supporting and adjusting fissile, and the apparatus includes components and embodiments for supporting and adjusting the tissue. Some embodiments include a supporting device, having a first portion, a second portion, and a support member positioned between the first portion and second portion. Some embodiments include advancing the first portion of the supporting device into the body to a first location in the body; advancing the second portion of the supporting device into the body to a second location in the body; securing the first portion of the supporting device at the first location; and shifting soft tissue in the body with the support member.

United States Patent Application 2009/0287227 to Newell, et al. discloses methods, systems, devices and assemblies for treating a patient by: making an incision or puncture though the patient's skin over the abdominal cavity. An initial tract is established through an opening formed by the incision or puncture. An instrument is advanced through the tract; contacting a distal end portion of the instrument against an inner surface of the abdominal cavity, and driving at least one stitching needle through the inner surface of the abdominal cavity. The driving is continued until the at least one stitching needle exits the inner surface of the abdominal cavity. A suture anchor is carried by each of the at least one stitching needle to a suture anchor at an exit location, respectively. Tension is applied to each of the sutures.

United States Patent Application 2010/0101526 to Bennett discloses a surgical suture system, suture, and tissue engaging member for tissue repair and reattachment of torn tissue to a tissue substrate, medical prosthesis or medical implant. The system includes the elongated flexible suture member having a plurality of longitudinally spaced protuberances along a length thereof and one or a plurality of the tissue engaging members each of which include two closely spaced apart locking apertures sized and configured to receive one of the suture members passed therethrough or a unique single locking aperture to allow longitudinal tensioning and/or restraining movement of the suture member in only one direction through the locking apertures for suture member tightening.

United States Patent Application 2010/0160963 to Fallin, et al. discloses a system for attaching soft tissue to bone including an anchor, a suture, and a tissue retainer. The anchor may have a threaded tip that engages the bone, and a suture retention portion with passageways arranged such that each of first and second anchor portions of the suture can be drawn through the passageways along only a single direction. The tissue retainer has passageways through which the suture can freely move in either direction. Thus, the anchor and the tissue retainer may be attached to hone and tissue, respectively, and the suture may be drawn to substantially irreversibly draw the hone and tissue together. In alternative embodiments, an anchor may receive only one portion of suture and/or one suture end may be affixed to the anchor. The anchor may alternatively permit free motion of the suture, while the tissue retainer permits passage of the suture along only one direction.

United States Patent Application 2012/0101524 to Bennett discloses a surgical suture system for tissue repair and reattachment of torn tissue to a tissue substrate, medical prosthesis or medical implant. The system includes an elongated flexible suture member having a plurality of longitudinally spaced protuberances along a length thereof and a plurality of tissue engaging members each including two spaced apart locking apertures sized to receive the suture member passed therethrough to allow longitudinal movement of the suture member in only one direction through the locking apertures for suture member tightening.

United States Patent Application 2013/0090686 to Covidien discloses barbed surgical sutures which include an elongated body and a plurality of barbs extending therefrom. A bioactive agent is disposed within barb angles formed between the barbs and the elongated body. The barbs may be made from a shape memory polymer having a permanent shape which may be deformed to a temporary shape, such that barbs of the suture extend at different barb angles in the different shape configurations. The barb angles of the permanent shape may be greater than the barb angles of the temporary shape, thereby exposing and/or releasing a bioactive agent after placement in tissue.

Although the forgoing prior art has contributed to the surgical art, it is a primary object of the present invention to provide a new system for securing sutures to a tissue or a bone suitable for use in general as well as arthroscopic, endoscopic, laparoscopic or minimally invasive surgery.

Therefore, it is an object of the present invention to provide an apparatus and method for securing flexible member including a method tier pushing a capture or a retention device down a flexible member such as a suture or the like.

Another object of this invention is to provide a novel method for securing sutures to a bone or a tissue or for securing a first tissue to a second tissue.

Another object of this invention is to provide a novel retention device capable of attaching to a flexible member such as a suture or the like.

The foregoing has outlined some of the more pertinent objects of the present invention. These objects should be construed as being merely illustrative of some of the more prominent features and applications of the invention. Many other beneficial results can be obtained by modifying the invention within the scope of the invention. Accordingly other objects in a full understanding of the invention may be had by referring to the summary of the invention, the detailed description describing, the preferred embodiment in addition to the scope of the invention defined by the claims taken in conjunction with the accompanying drawings.

### SUMMARY OF THE INVENTION

The present invention is defined by the appended claims with specific embodiments being shown in the attached drawings. For the purpose of summarizing the invention, the first embodiment of the invention relates to a method of securing a suture comprising the steps of providing a retention device having an aperture. The suture is passed through the aperture in the retention device. A portion of the retention device is deformed to secure the suture to the retention device.

A second embodiment of the invention relates to a method of securing a suture comprising the steps of providing a retention device having an aperture. The suture is inserted through the aperture in the retention device. The retention device is moved along the suture to position the retention device at a desired location along the suture. A portion of the retention device is deformed to secure the suture to the retention device.

A third embodiment of the invention relates to a method of securing a tissue, comprising the steps of providing an anchor having an anchor aperture. A suture retention device is attached in proximity to a first end of a suture. A second end of the suture is passed through the tissue. The second end of the suture is inserted through the anchor aperture. The second end of the suture is pulled to tighten the suture retention device against the tissue. The second end is secured.

In a more specific example of the invention, the step of providing an anchor comprises providing an anchor having a one-way anchor lock. The step of securing the suture to the anchor comprises pulling the second end of the suture through the one-way anchor lock for securing the suture in the anchor. The step of attaching a suture retention device in proximity to a first end of a suture includes the step of crimping the suture retention device to the suture. The step of attaching a suture retention device in proximity to a first end of a suture includes the step of rotationally securing the suture retention device to the suture.

A fourth embodiment of the invention relates to a method of securing a tissue, comprising the steps of providing an anchor having an anchor aperture. The first end of the suture is passed through a first aperture in a first suture retention device. A first suture fastener is secured to the suture adjacent to the first suture retention device. A second end of the suture is passed through the tissue. The second end of the suture is inserted into the anchor aperture. The second end of the suture is passed through a second aperture in a second suture retention device. The second end of the suture is pulled to tighten the first suture retention device against the tissue. A second suture fastener is secured to the suture adjacent to the second suture retention device.

In as more specific example of the invention, the step of securing a first suture fastener to the suture includes the step of crimping the first suture fastener to the suture. The step of securing a first suture fastener to the suture includes the step of rotationally securing the first suture fastener to the suture.

A fifth embodiment of the invention relates to a method of securing a first tissue to a second tissue, comprising the steps of passing a first end of the suture through a first aperture in a first suture retention device. A first suture fastener is secured to the suture adjacent to the first suture retention device. A second end of the suture is passed through the first tissue. The second end of the suture is passed through the second tissue. The second end of the suture is passed through a second aperture in a second suture retention device. The second end of the suture is pulled to tighten the first suture retention device against the first tissue and the second suture retention device against the second tissue. A second suture fastener is secured to the suture adjacent to the second suture retention device.

In a more specific example of the invention, the step of securing a first suture fastener to the suture includes the step of crimping the first suture fastener to the suture. The step of securing a first suture fastener to the suture includes the step of rotationally securing the first suture fastener to the suture

A sixth embodiment of the invention relates to a method of securing a flexible member comprising the steps of providing a first member having a first orifice. A second member has a second orifice. The second member is located relative to the first members with the second orifice aligned with the first second orifice for enabling the flexible member to extend through the first and second orifices. The second member is deformed for securing the flexible member.

A seventh embodiment of the invention relates to a method of securing a flexible member, comprising the steps of providing a first member having a first orifice. A second member has a second orifice. The second member is located relative to the first members with the second orifice aligned with the first second orifice for enabling the flexible member to extend through the first and second orifices. The second member is rotated relative to the first member for securing the flexible member.

An eighth embodiment of the invention relates to a retention device for securing a flexible member comprising a first member having a first orifice. A second member has a second orifice. The first member is disposed relative to the second members with the first orifice aligned with the second orifice for enabling the flexible member to extend through the first and second orifices. The second member is deformable for securing the flexible member.

In a more specific example of the invention, the second member is integral with the first member. The second member is moveable relative to the first member for securing the flexible member. The second member is moveable relative to the first member for crimping the flexible member.

A ninth embodiment of the invention relates to a retention device for securing a flexible member comprising a first member having a first orifice. A second member has a second orifice. A rotational mounting rotatably secures the second member relative to the first member with the first orifice aligned with the second orifice for enabling the flexible member to extend through the first and second orifices when the first and second members are disposed in a first rotational position. The rotational mounting enables relative rotation of the first and second members into a second rotational position for securing the flexible member.

In a more specific example of the invention, the second member is rotatable relative to the first member for crimping the flexible member. The first threads are located on the first member and the second threads are located on the second member enabling the second member to threadably engage for crimping to the flexible member. First threads are located on the first member and second threads are located on the second member enabling the second member to he threadably rotated on to the first member for deforming the first member into engagement with the flexible member.

A tenth embodiment of the invention relates to a retention device for securing a flexible member comprising a first member having a first orifice. A second member has a second orifice. A rotational mounting rotatably mounts the second member relative to the first member with the first orifice aligned with the second orifice for enabling the flexible member to extend through the first and second orifices when the first and second members are disposed in a first rotational position. A lock fastens the second member in a second rotational positron relative to the first member whereat the first orifice is misaligned with the second orifice for securing the flexible member between the first and second members.

In a more specific example of the invention, the first member is an outer member and the second member is an inner member. A retainer retains the rotatable inner member relative to the outer member. The rotational mounting includes a recess defined in the first member for receiving the second member for rotatably mounting the second member relative to the recess. The lock includes a projection located on one of the first and second members cooperating with a depression located in the other of the first and second members for maintaining the second member in the second position. The lock comprises a ratchet interacting between the first and second members for maintaining the second member in the second rotational position.

The foregoing has outlined rather broadly the more pertinent and important features of the present invention in order that the detailed description that follows may be better understood so that the present contribution to the art can be more fully appreciated. Additional features of the invention will be described hereinafter which firm the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a fuller understanding of the nature and objects of the invention, reference should be made to the following detailed description taken in connection with the accompanying drawings in which:

FIG. 1 illustrates a first step in a first method of securing a tissue to a bone including providing an anchor in the bone and attaching a suture retention device in proximity to a first end of the suture;

FIG. 2 illustrates a second step in securing the tissue to the bone including passing the second end of the suture through the tissue;

FIG. 3 illustrates a third step in securing the tissue to the bone including inserting the second end of the suture into the anchor;

FIG. 4 illustrates a fourth step in securing the tissue to the bone including pulling the second end of the suture to tighten the suture retention device against the tissue enabling the securing of the suture;

FIG. 5 illustrates a first step in a second method of securing a tissue including providing an anchor having an anchor aperture.

FIG. 6 illustrates a second step in securing the tissue to the bone including passing a first end of the suture through a first orifice in a first suture retention device and securing a first suture fastener to the suture adjacent to the first suture retention device;

FIG. 7 illustrates a third step in securing the tissue to the bone including passing a second end of the suture through the tissue;

FIG. 8 illustrates a fourth step in securing the tissue to the bone including inserting the second end of the suture into the anchor aperture;

FIG. 9 illustrates a fifth step in securing the tissue to the bone including passing the second end of the suture through a second orifice in a second suture retention device and pulling the second end of the suture to tighten the first suture retention device against the tissue;

FIG. 10 illustrates a sixth step in securing the tissue to the bone including pushing the second suture retention device into engagement with the tissue;

FIG. 11 illustrates a seventh step in securing the tissue to the bone including securing a second suture fastener to the suture adjacent to the second suture retention device;

FIG. 12 illustrates a first step in a third method of securing a tissue including passing a first end of the suture through a first orifice in a first. suture retention device and securing a first suture fastener to the suture adjacent to the first suture retention device then passing a second end of the suture through the first tissue and the second tissue;

FIG. 13 illustrates a second step in a third method of securing a tissue including passing the second end of the suture through a second orifice in a second suture retention device and pulling the second end of the suture to tighten the first suture retention device against the first tissue and the second suture retention device against the second tissue and then securing a second suture fastener to the suture adjacent to the second suture retention device;

FIG. 14 illustrates a first device for securing a suture fastener to the suture;

FIG. 15 illustrates the first device of FIG. 14 pushing a suture retention device;

FIG. 16 illustrates the first device of FIGS. 14 and 15 securing the suture fastener to the suture by crimping the suture fastener to the suture;

FIG. 17 illustrates a second device for securing a suture fastener to the suture;

FIG. 18 illustrates the second device of FIG. 17 pushing a suture retention device;

FIG. 19 illustrates the second device of FIGS. 17 and 18 securing the suture fastener to the suture by compressing the suture fastener to the suture by a threaded engagement;

FIG. 20 is an isometric view of a third retention device for securing to a flexible member such as a suture in a first rotational position;

FIG. 21 is a view similar to FIG. 20 with the third retention device in a second rotational position;

FIG. 22 is a top view of FIG. 20;

FIG. 23 is a sectional view along line 23-23 in FIG. 22;

FIG. 24 is a view similar to FIG. 22 with the third retention device in a second rotational position;

FIG. 25 is a sectional view along line 25-25 in FIG. 24;

FIG. 26 is an isometric view of a first or outer member of the third retention device;

FIG. 27 is a top view of FIG. 26;

FIG. 28 is a sectional view along line 28-28 in FIG. 27;

FIG. 29 is an isometric view of a second or inner member of the third retention device;

FIG. 30 is a top view of FIG. 29;

FIG. 31 is a sectional view along line 31-31 in FIG. 30;

FIG. 32 is an isometric view of a fourth retention device for securing to a flexible member such as a suture in a first rotational position;

FIG. 33 is a view similar to FIG. 32 with the fourth retention device in a first rotational position;

FIG. 34 is a top view of FIG. 32;

FIG. 35 is a sectional view along line 35-35 in FIG. 34;

FIG. 36 is a sectional view along line 36-36 in FIG. 34;

FIG. 37 is a top view of FIG. 33;

FIG. 38 is a sectional view along line 38-38 in FIG. 37;

FIG. 39 is a sectional view along line 39-39 in FIG. 37;

FIG. 40 is an isometric view of a first or outer member of the fourth retention device;

FIG. 41 is a top view of FIG. 40;

FIG. 42 is a sectional view along line 42-42 in FIG. 41;

FIG. 43 is an isometric view of a second or inner member of the fourth retention device;

FIG. 44 is a top view of FIG. 43;

FIG. 45 is a sectional view along line 45-45 in FIG. 44;

FIG. 46 is a side view of a device for securing the retention device of FIGS. 20-45 to a flexible member;

FIG. 47 is a side sectional view of the device of FIG. 46;

FIG. 48 is a left end view of the device of FIG. 47;

FIG. 49 is a right end view of the device of FIG. 47;

FIG. 50 is a magnified view of a portion of FIG. 47;

FIG. 51 is a first step in the process of securing the retention device of FIGS. 20-45 to the flexible member illustrating the threading of the retention device onto the flexible member;

FIG. 52 is a second step in the process of securing the retention device of FIGS. 20-45 to the flexible member illustrating the pushing of the retention device along the flexible member;

FIG. 53 is a bottom view of the device of FIG. 52;

FIG. 54 is a third step in the process of securing the retention device of FIGS. 20-45 to the flexible member illustrating the pushing of the retention device along the flexible member; and

FIG. 55 is a bottom view of the device of FIG. 54.

Similar reference characters refer to similar parts throughout the several Figures of the drawings.

### DETAILED DISCUSSION

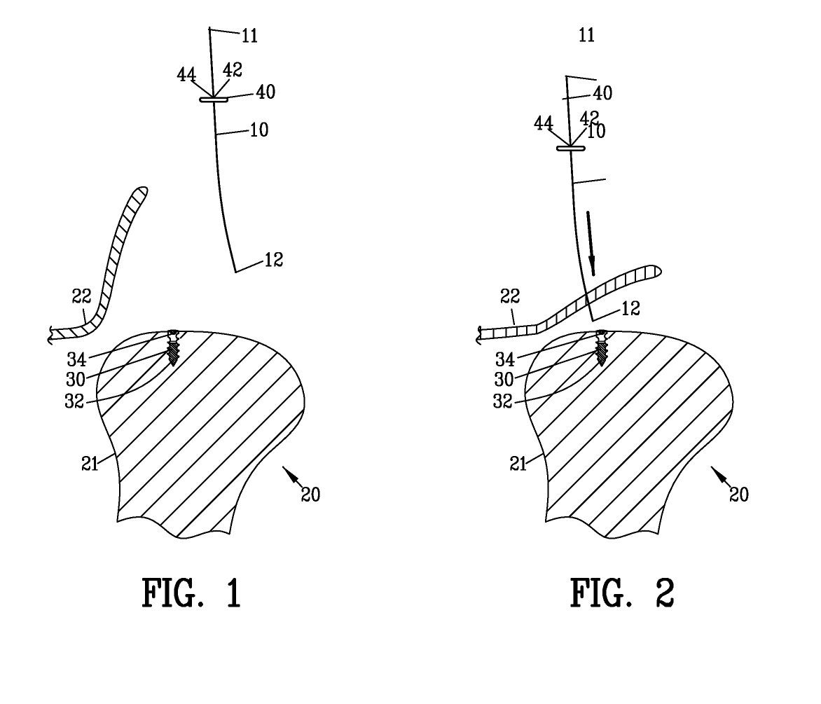

FIGS. 1-4 illustrate a first method of using a flexible member such as a suture 10 for securing a tissue 20. The suture 10 extends between a first end ii and a second end 12. in this example, the tissue 20 comprises a bard tissue shown as a bone 21 and a soft tissue 22.

FIG. 1 illustrates a first step in the first method of securing a soft tissue 22 to the bone 21 including providing an anchor 30 in the bone 21. The anchor 30 is shown having a screw body 32 and an anchor aperture 34. Although the anchor 30 has been shown as a screw type anchor, it should be understood that virtually any type of anchor may be used with the present method.

A suture retention device 40 has a suture orifice 42 and a retention lock 44. The first end of the suture 10 is threaded through the suture orifice 42 and is fixed in position on the suture 10 by the retention lock 44. The first end of the suture 10 may be fixed to the suture 10 by the retention lock 44 during a manufacturing process or may be affixed during a surgical procedure.

FIG. 2 illustrates a second step in securing a tissue 22 to the bone 21 including passing the second end 12 of the suture 10 through the tissue 22. The second end 12 of the suture 10 is passed through the tissue 22 using suitable means (not shown) which should be well known to those skilled in the surgical art.

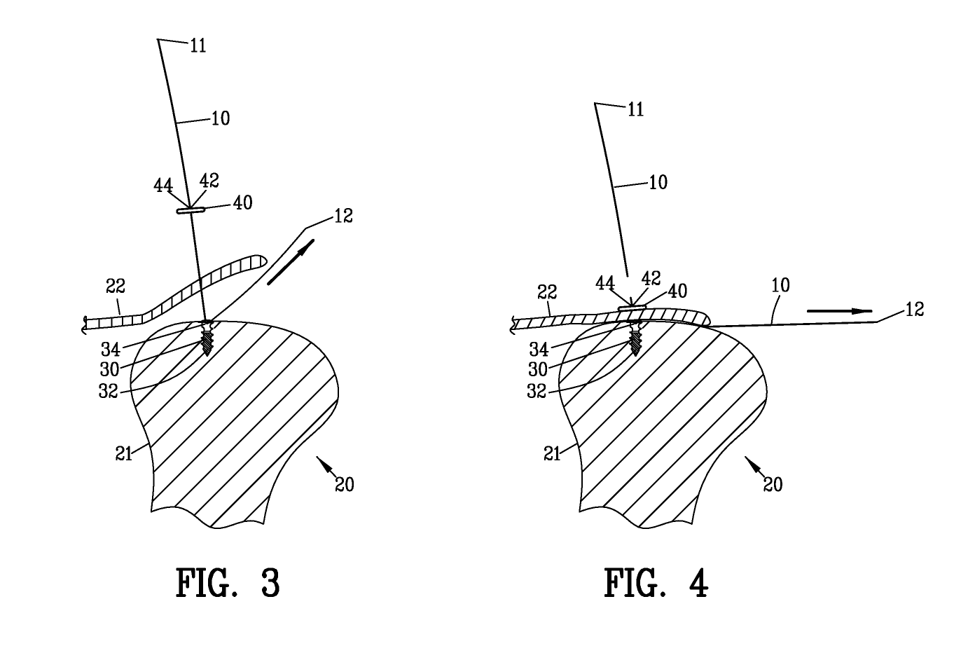

FIG. 3 illustrates a third step in securing a tissue 22 to the bone 21 including inserting the second end 12 of the suture 10 into the anchor aperture 34 of the anchor 30. The second end 12 of the suture 10 may be inserted into the anchor aperture 34 of the anchor 30 using suitable means such as a shuttle (not shown) which should be well known to those skilled in the surgical art.

FIG. 4 illustrates a fourth step in securing a tissue 22 to as bone 21 including pulling the second end 12 of the suture 10 to tighten the suture retention device 40 against the tissue 22 and the tissue 22 against the bone 21. The second end 12 of the suture 10 may be fastened in a conventional fashion to retain the tissue 22 against the bone 21. The remainder of the first end 11 of the suture 10 is severed from the second end 12 of the suture 10.

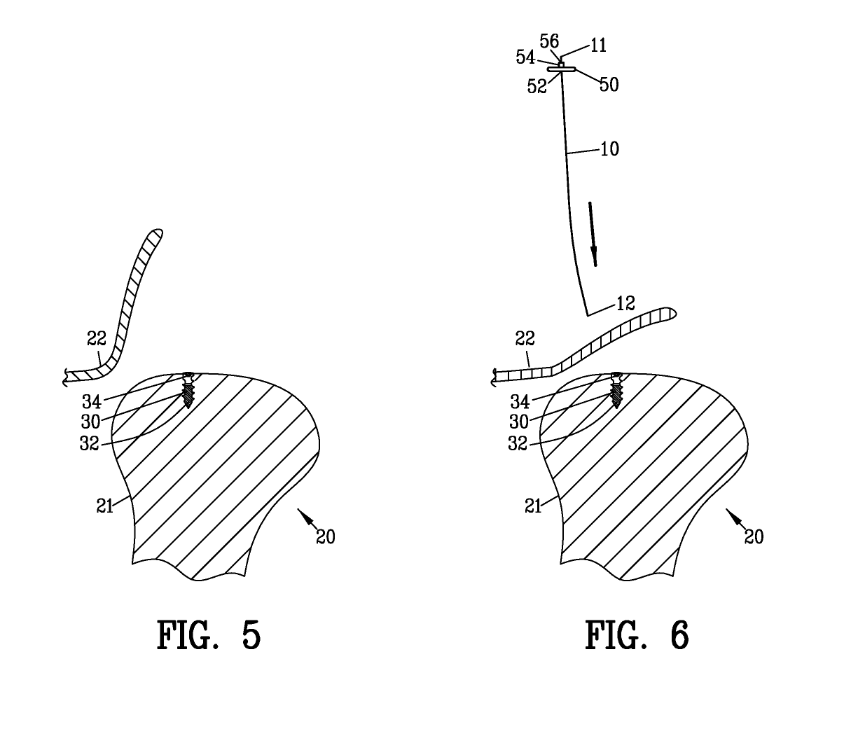

FIG. 5 illustrates a first step in a second method of securing a tissue 21 to a bone 21 including providing an anchor 30 having an anchor aperture 34. Virtually any type of anchor may be used with the present method.

FIG. 6 illustrates a second step in securing the tissue 22 to the bone 21 including passing a first end 11 of the suture 10 through a first orifice 52 in a first suture retention device 50 and through a fastener orifice 56 in a first suture fastener 54. The first suture fastener 54 is secured to the suture 10 adjacent to the first suture retention device 50. The first end of the suture 10 may be affixed to the suture 10 during a manufacturing process or may be affixed during a surgical procedure.

In this example, the suture fastener 54 is a deformable member far securing to the suture 10 upon deformation of the suture fastener 54. Preferably, the suture fastener 54 is affixed to the suture retention device 50. In the alternative, the suture fastener 54 is separate part from the suture retention device 50.

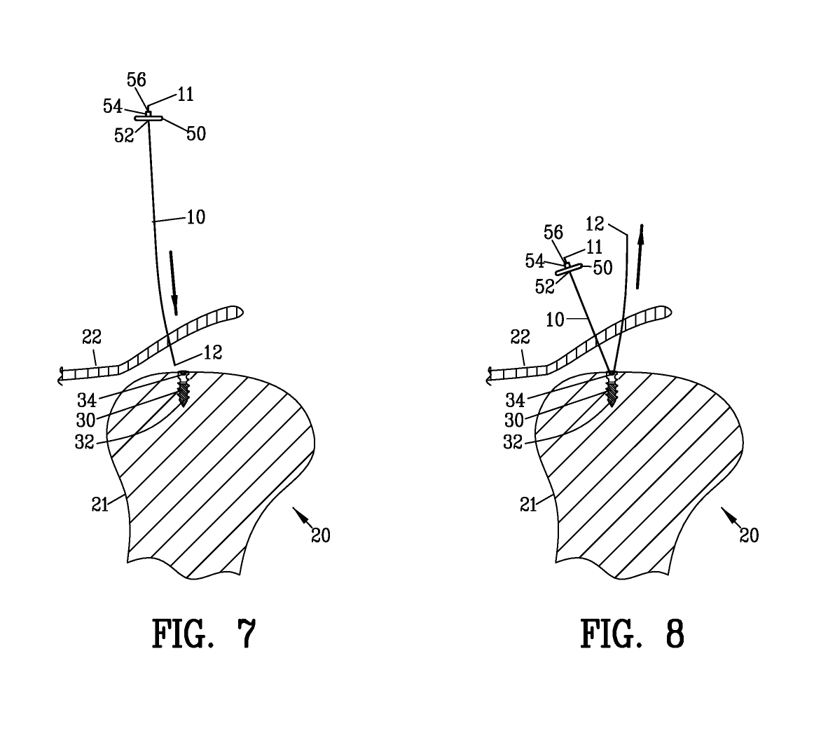

FIG. 7 illustrates a third step in securing the tissue 22 to the bone 21 including passing a second end 12 of the suture 10 through the tissue 22. The second end 12 of the suture 10 is passed through the tissue 22 using suitable means (not shown) which should be well known to those skilled in the surgical art.

FIG. 8 illustrates a fourth step in securing the tissue 22 to the bone 21 including inserting the second end 12 of the suture 10 into the anchor aperture 34. Virtually any type of anchor may be used with the present method. The second end 12 of the suture 10 is again passed through the tissue 22 to extend from the upper side of the tissue 22.

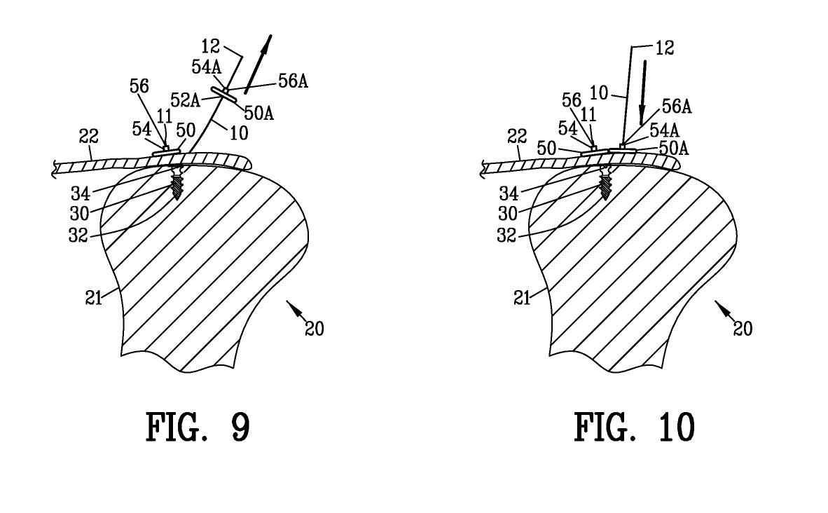

FIG. 9 illustrates a fifth step in securing the tissue 22 to the bone 21 including passing the second end 12 of the suture 10 through a second orifice 52A in a second suture retention device 50A and passing second end 12 of the suture 10 through a second orifice 56A in a second suture fastener 54A.

The second end 12 of the suture 10 is pulled to tighten the first suture retention device 50 against the tissue 22 and to tighten the tissue 22 against the bone 21. The movement of the second end 12 of the suture 10 is effected by suitable means (not shown) which should be well known to those skilled in the surgical art.

FIG. 10 illustrates a sixth step in securing the tissue 22 to the bone 21 pushing the second suture retention device 50A into engagement with the tissue 22. The second suture retention device 50A is pushed into engagement with the tissue 22 simultaneously with the second suture fastener 54A. The preferred method of pushing the second suture retention device 50A simultaneously with the second suture fastener 54A will be set forth in greater detail hereinafter.

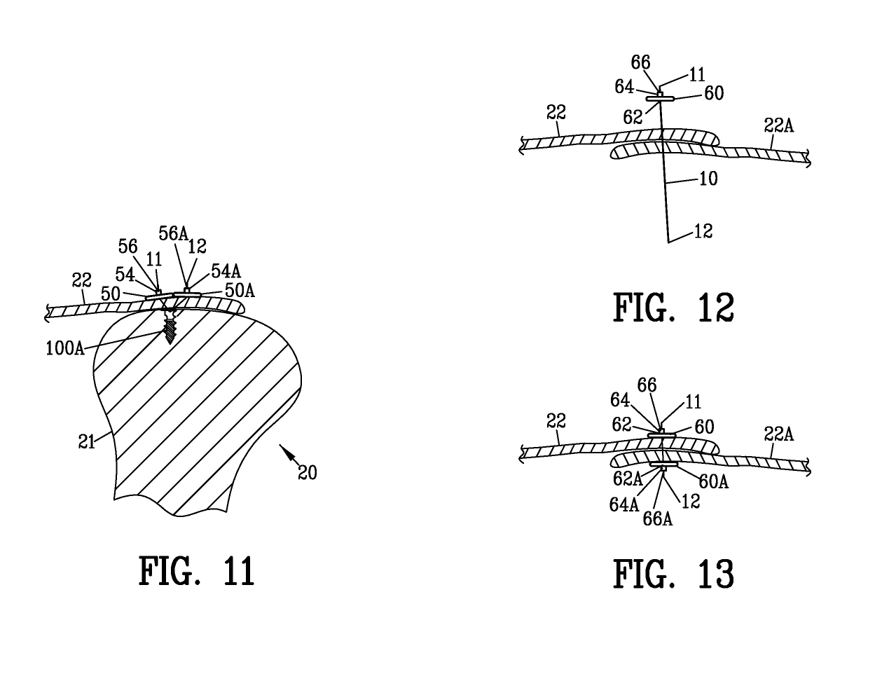

FIG. 11 illustrates a sixth step in securing the tissue 22 to the bone 21 including securing a second suture fastener 54A to the suture 10 adjacent to the second suture retention device 50A. The second suture fastener 54A is fixed to the suture 10 adjacent to the second suture retention device 50A upon deformation of the second suture fastener 54A. The first and second suture retention devices 50 and 50A maintain engagement of the tissue 22 with the bone 21.

FIG. 12 illustrates a first step in a third method of securing a tissue including passing a first end 11 of the suture 10 through a first orifice 62 in a first suture retention device 60 and passing the first end 11 of the suture 10 through a first orifice 66 in a first suture fastener 64. The first suture fastener 64 is secured to the suture 10 adjacent to the first suture retention device 60. The second end 12 of the suture 10 is passed through a first tissue 22 and a second tissue 22A.

FIG. 13 illustrates a second step in the third method of securing a tissue including passing the second end 12 at the suture 10 through a second orifice 62A in a second suture retention device 60A and passing the second end 12 of the suture 10 through a second orifice 66A in a second suture fastener 64A. The second end 12 of the suture 10 is pulled to tighten the first suture retention device 60 against the first tissue 22.

The second suture retention device 60A is pushed into engagement with the tissue 22A along with the second suture fastener 64A. The second suture fastener 64A is fixed to the suture 10 adjacent to the second suture retention device 60A upon deformation of the second suture fastener 64A. The first and second suture retention devices 60 and 60A maintain engagement between the tissues 22 and 22A.

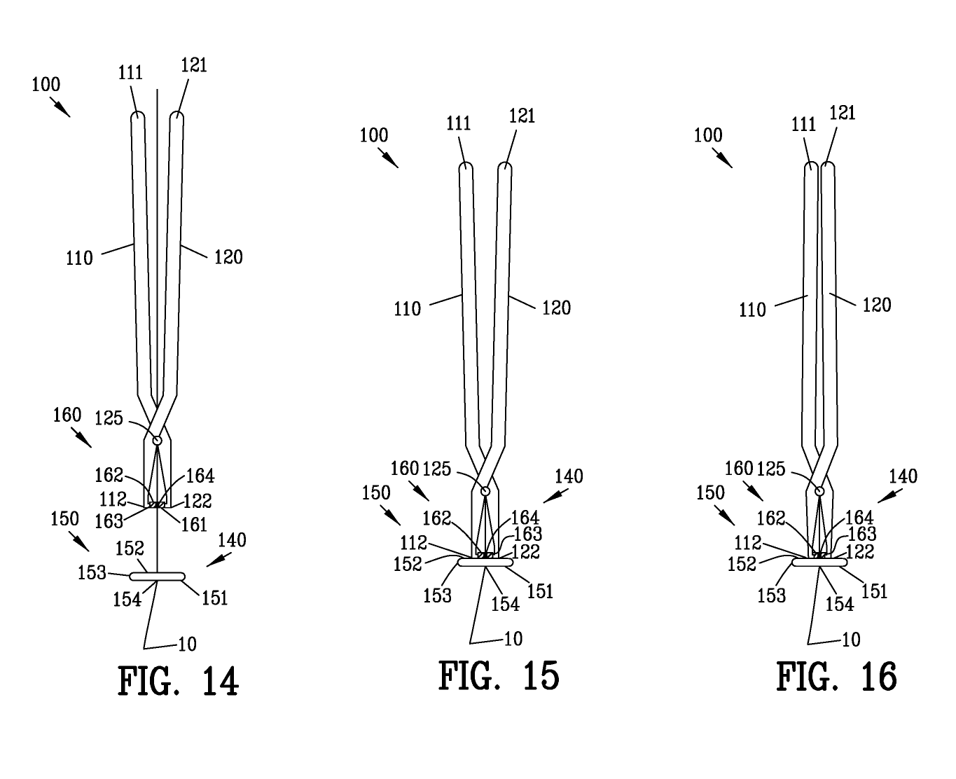

FIGS. 14-16 illustrate a first embodiment of a device 100 suitable for accomplishing the methods set forth in FIGS. 1-13. The first embodiment of a device 100 comprises a first and a second arm 110 and 120. The first arm 110 extends between a proximal end 111 and a distal end 112. Similarly, the second arm 120 extends between a proximal end 121 and a distal end 122. The first and second arms 110 and 120 are connected by a pivot 125 intermediate the proximal ends 111 and 121 and the distal end 112 and 122. The proximal ends 111 and 121 function as a handle to open and close the distal ends 112 and 122 that function as jaw.

In this example, the suture retention device 140 comprises a first member 150 and a second member 160. The first member and second members 150 and 160 may be representative of the suture retention device 50 and the suture fastener 54 in FIGS. 5-11.

The first member 150 defines a lower surface 151 and an upper surface 152 and a peripheral edge 153. A first orifice 154 extends between the lower surface 151 and the upper surface 152. The second member 160 defines a lower surface 161 and an upper surface 162 and a peripheral edge 163. A first orifice 164 extends between the lower surface 161 and the upper surface 162. In this example the first member 150 is separate from the second member 160.

The second member 160 is located relative to the first members 150 with the second orifice 164 aligned with the first orifice 154 for enabling the flexible member 10 to extend through the first and second orifices 154 and 164.

FIG. illustrates the device 100 grasping the second member 160 with the suture 10 extending through the second orifice 164 in the second member 160. A moderate compression inward force applied to the proximal ends 111 and 121 enable the distal end 112 and 122 to grasp the peripheral edge 163 of the second member 160.

FIG. 15 illustrates the first device 100 of FIG. 14 pushing the first member 150 while grasping the second member 160. The distal end 112 and 122 of the first and second arm 110 and 120 push the first and second members 150 and 160 along the suture 10.

FIG. 16 illustrates the first device 100 of FIGS. 14 and 15 securing the second member 160 to the suture 10. A strong compression inward three applied to the proximal ends 111 and 121 enable the distal end 112 and 122 to compress the peripheral edge 163 of the second member 160 and to permanently deform the second orifice 164 to securing the second member 160 to the suture 10. The permanent deformation of the second member 160 pinches or crimps second member 160 to the suture 10. Thereafter, the device 100 may be removed from the second member 160.

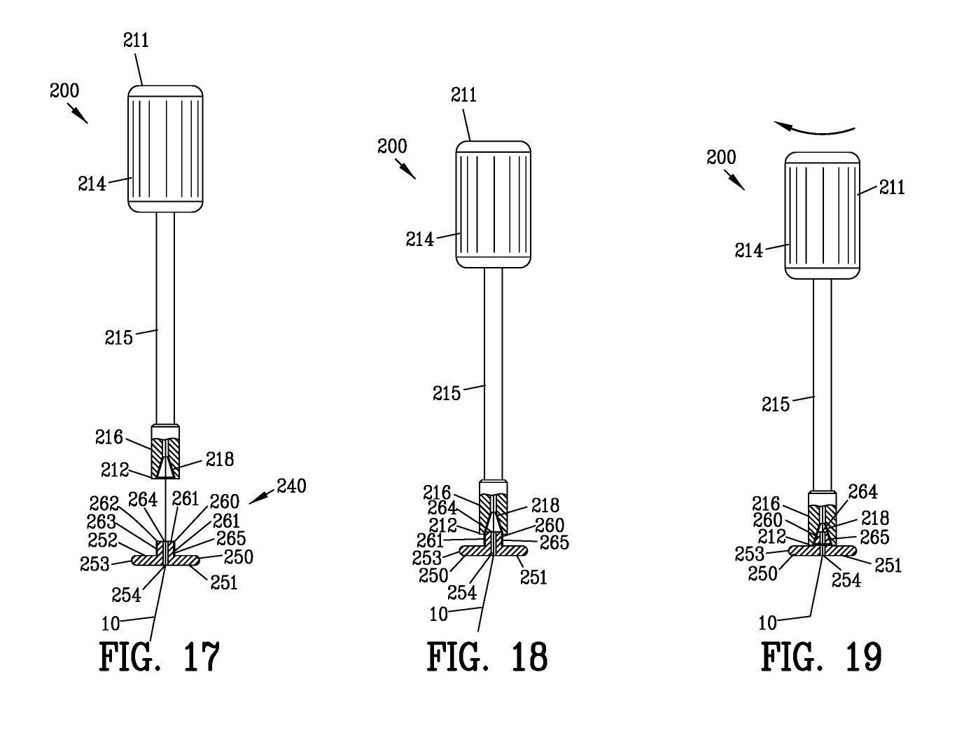

FIGS. 17-19 illustrate a second embodiment of a device 200 suitable for accomplishing the methods set forth in FIGS. 1-13. The second embodiment of a device 200 extends between a proximal end 211 and a distal end 212. The proximal end 211 functions as a handle 214 to rotate the device 200. A shaft 215 is interposed between the proximal end 211 and the distal end 212. The distal end 212 comprises a head 216 having internal treads 218. In this example, the internal treads 218 are formed in a shape of a cone.

In this example, the suture retention device 240 comprises a first member 250 and a second member 260. The first member and second members 250 and 260 may be representative of the suture retention device 50 and the suture fastener 54 in FIGS. 5-11.

The first member 250 defines a lower surface 251 and an upper surface 252 and a peripheral edge 253. A first orifice 254 extends between the lower surface 251 and the upper surface 252. The second member 260 defines a boss 261 extending from the upper surface 252 to an upper surface 262. The boss 261 defines a peripheral edge 263 having external threads 265. A second orifice 264 extends through the boss 261 and is aligned with the first orifice 254 for enabling the flexible member 10 to extend therethrough. In this example, the first and second members 250 and 260 are formed as a one piece unit.

FIG. 17 illustrates the device 200 spaced from the first and second members 250 and 260 with the suture 10 extending through orifices 254 and 264. The suture 10 extends through the shaft 215 of the device 200.

FIG. 18 illustrates the second device 200 of FIG. 17 pushing the second member 260 along the suture 10. The head 216 engages the external threads 265 of the boss 261 of the second member 260 to move the suture retention device 240 along the suture 10.

FIG. 19 illustrates the second device 200 of FIG. 18 securing the second member 260 to the suture 10. A rotation of the handle 214 threadably engages the internal treads 218 of the head 216 with the external threads 265 of the boss 261 of the second member 260 to permanently deform the boss 261 to secure the first and second members 250 and 260 to the suture 10. The rotational deformation of the boss 261 pinches or crimps second orifice 264 into engagement with the suture 10. Thereafter, the device 200 may be removed from the boss 261.

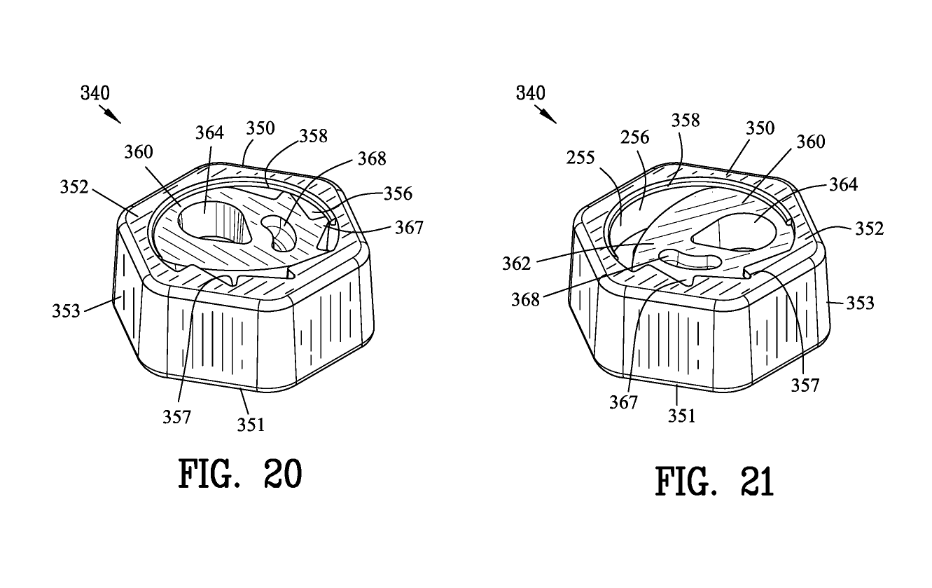

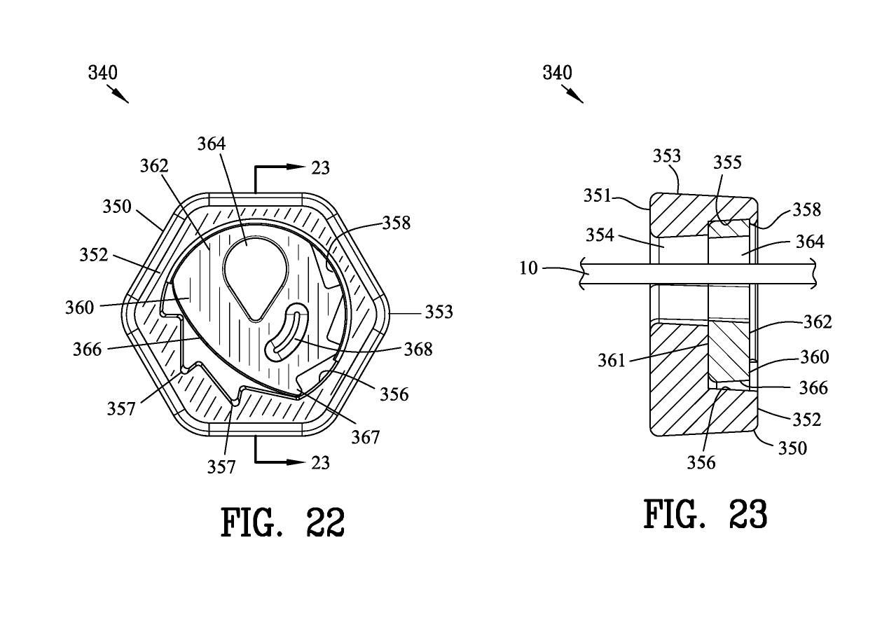

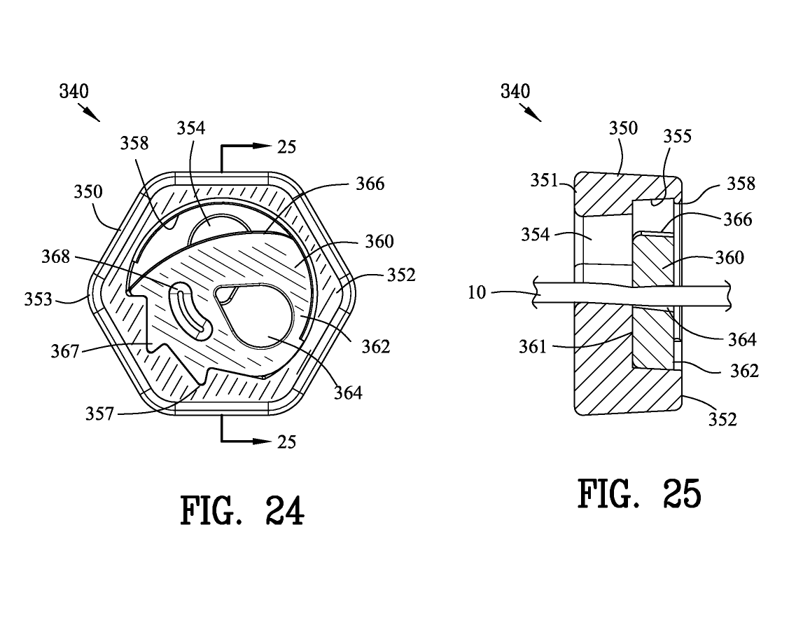

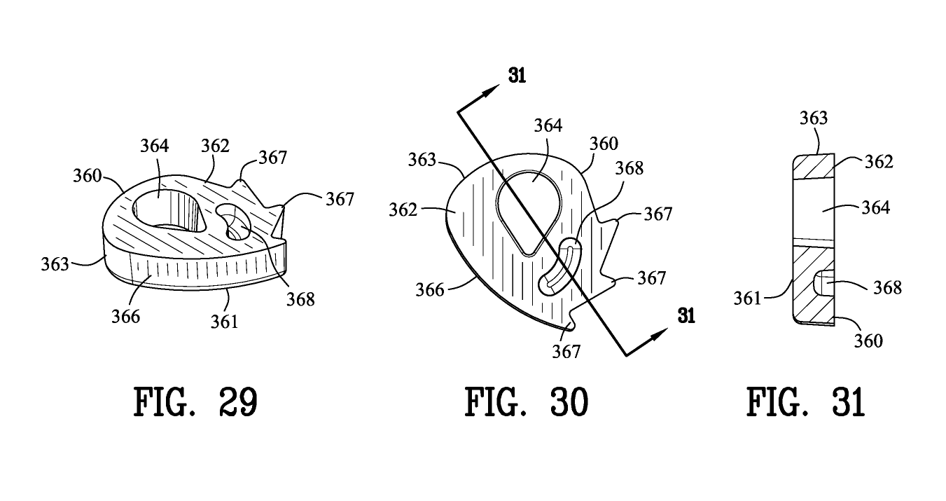

FIGS. 20-31 illustrate a third embodiment of a third retention device 340 suitable for securing to a flexible member such as a suture 10. The third embodiment of a third retention device 340 comprises a first member 350 and a second member 360.

FIGS. 20 and 22-23 is an isometric view of the third retention device 340 with the second member 360 in a first rotational position whereas FIGS. 21 and 24-25 illustrate the third retention device 340 with the second member 360 in a second rotational position. In this example, the first member 350 is an outer member and the second member is an inner member.

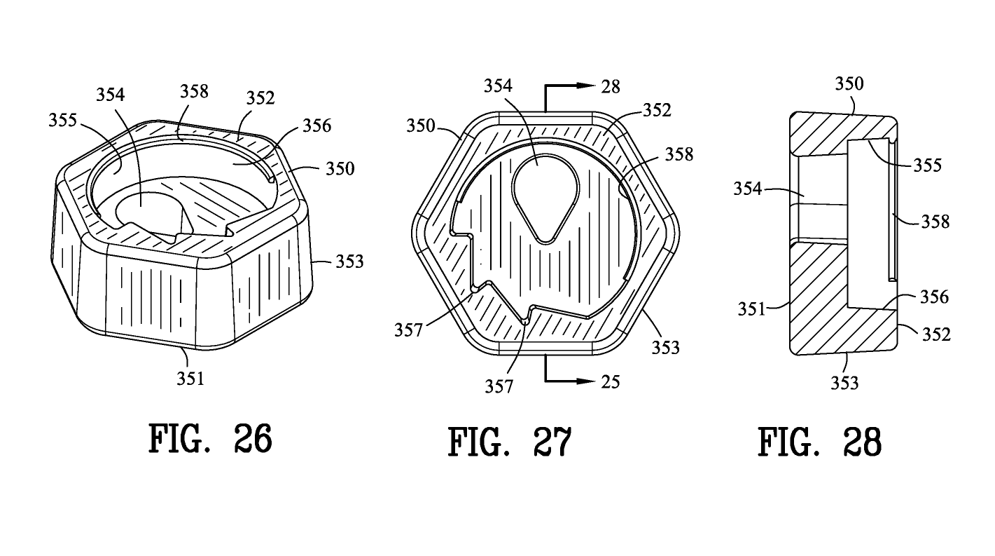

FIGS. 26-28 illustrate the first member 350 defining a lower surface 351 and an upper surface 352 and a peripheral edge 353. The peripheral edge 353 is formed in the shape of a hexagon for engaging with a socket of a tool as will be described in greater detail hereinafter. Although the peripheral edge 353 is formed in the shape of a hexagon, it should be understood that various other shapes may be utilized for cooperating with a tool as will be described hereinafter.

Recess 355 extends into the first member 350 from the upper surface 352 for rotatably receiving the second member 260. A first orifice 354 extends between the lower surface 351 and the recess 355.

The recess 355 defines a generally cylindrical sidewall 356 with plural depressions 357 extending into the generally cylindrical sidewall 356. A projection 358 extends radially inwardly from the generally cylindrical sidewall 356 for retaining the second member as will be describe in greater detail hereinafter.

The second member 360 defines a lower surface 361 and an upper surface 362 and a peripheral edge 363. A second orifice 364 extends between the lower surface 361 and the upper surface 362. The peripheral edge 363 is formed in the shape of a generally cylindrical sidewall 366 with plural teeth 367 extending from the generally cylindrical sidewall 366. A hollow 368 is defined in the upper surface 362 of the second member 360 the function of which will be described hereafter.

Referring back to FIGS. 20 and 22-23, the second member is shown rotatably mounted within recess of the first member 350 in the first rotational position. The first orifice 354 is aligned with the second orifice 364 for enabling the flexible member 10 to extend through the first and second orifices 354 and 364. The second member 360 is retained within the recess 355 by the projection 3589 engaging the upper surface 362 of the second member 360. The first and second members 350 and 360 are formed of a resilient material enabling the second members 360 to be inserted into the recess 355 past the projection 358. The teeth 367 of the second member 360 are compressed against the cylindrical sidewall 356 of the first member 350 in the first rotational position shown in FIGS. 20 and 22-23.

Referring back to FIGS. 21 and 24-25, the second member 360 is shown rotatably mounted within recess 355 of the first member 350 in the second rotational position. As best shown in FIG. 25, the second orifice 364 is misaligned with the first orifice 354 when the second member 360 is rotated relative to the first member 350 into the second position. The misalignment of the second orifice 364 relative to the first orifice 354 secures the flexible member 10.

Simultaneously, the teeth 367 of the second member 360 resiliently expand to engage with the depressions 357 in the first member 350. The depressions 357 of the lust member 350 and the teeth 367 of the second member 360 cooperate to function as a ratchet to lock the second member 360 in the second position relative to the first member 350.

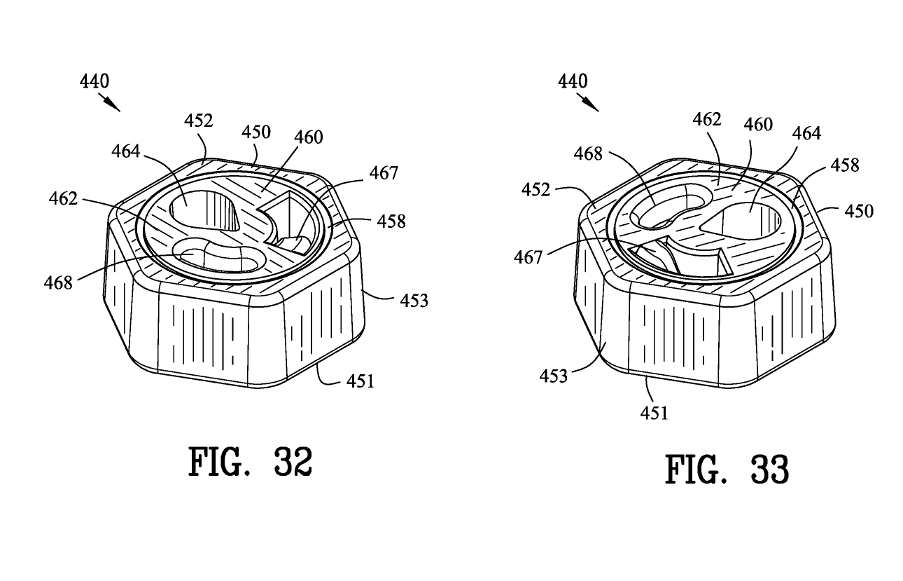

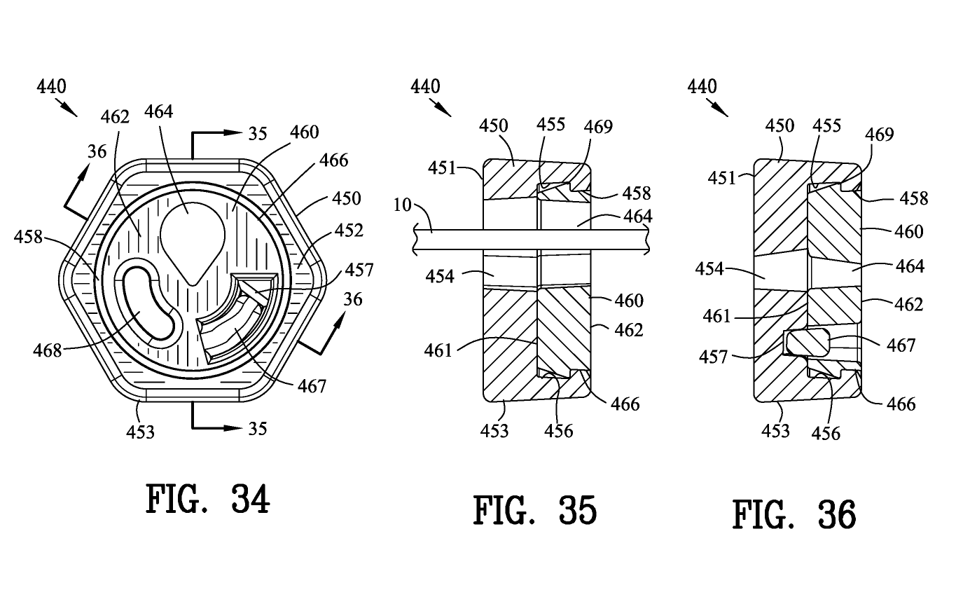

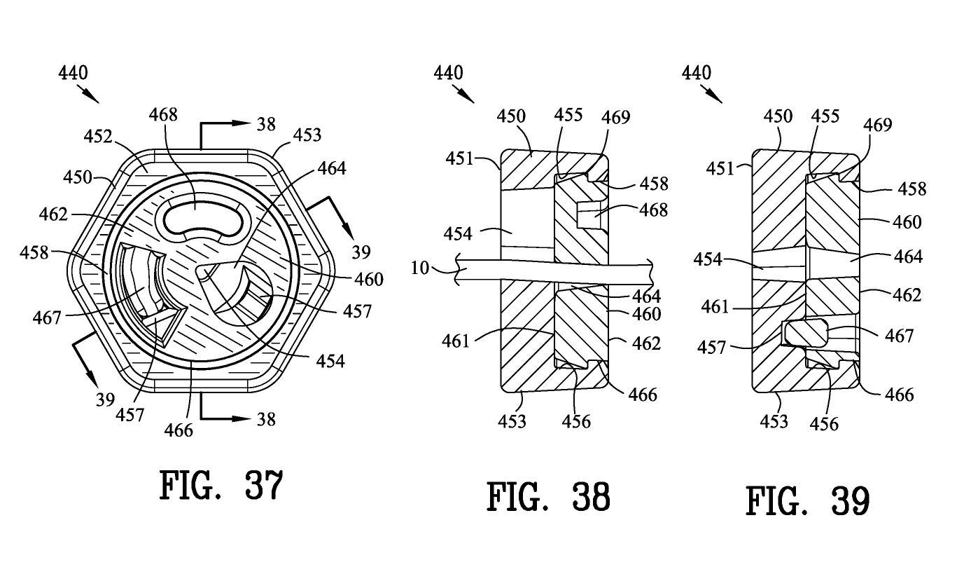

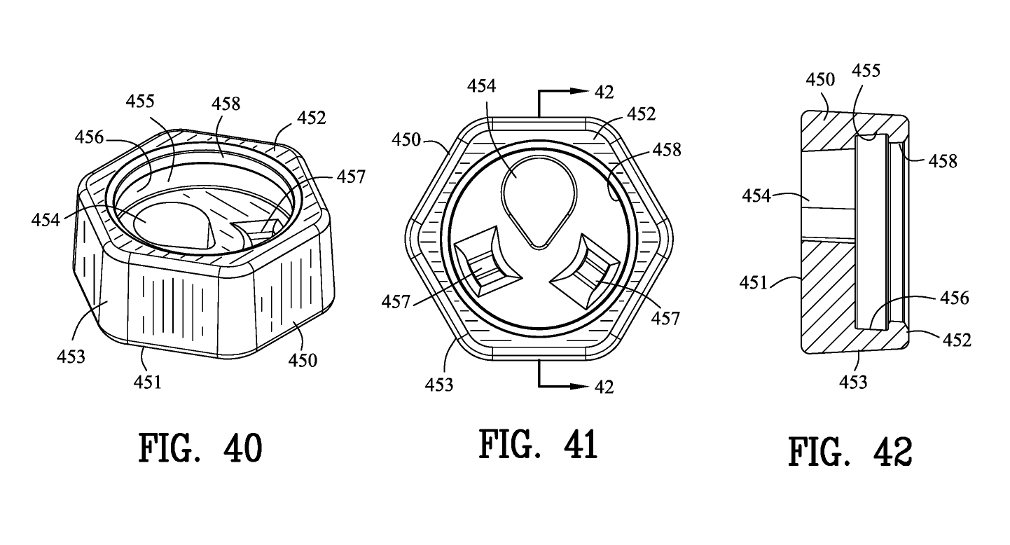

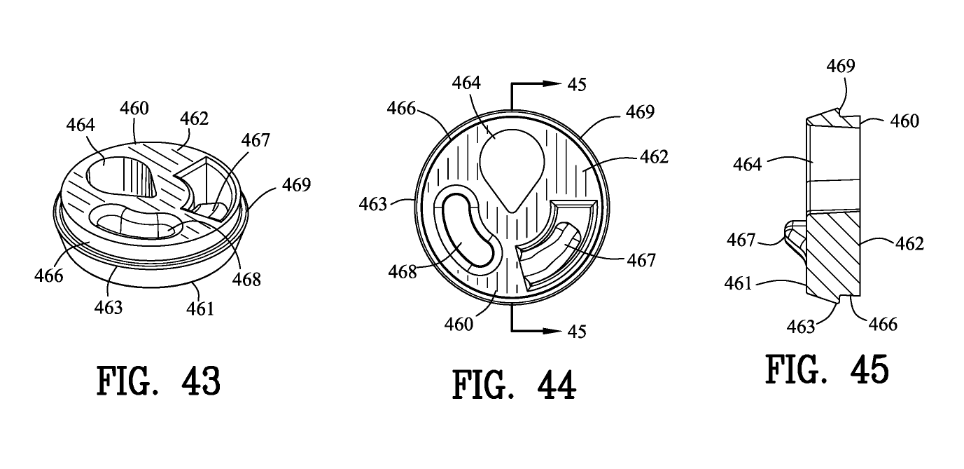

FIGS. 32-45 illustrate a fourth embodiment of a retention device 440 suitable for securing to a flexible member such as a suture 10. The fourth retention device 440 comprises a first member 450 and a second member 460.

FIGS. 32 and 34-36 is an isometric view of the fourth retention device 440 with the second member 460 in a first rotational position whereas FIGS. 33 and 37-39 illustrate the fourth retention device 440 with the second member 460 in a second rotational position.

The fourth retention device 440 is similar to the third retention device 340 with similar parts labeled with similar numbers raised by 100. In the fourth retention device 440, the teeth 467 are located on the lower surface 461 of the second member 460 for engagement with the depression defined on the recess 455 of the first member 450. The fourth retention device 440 operates in a manner similar to the third retention device 340.

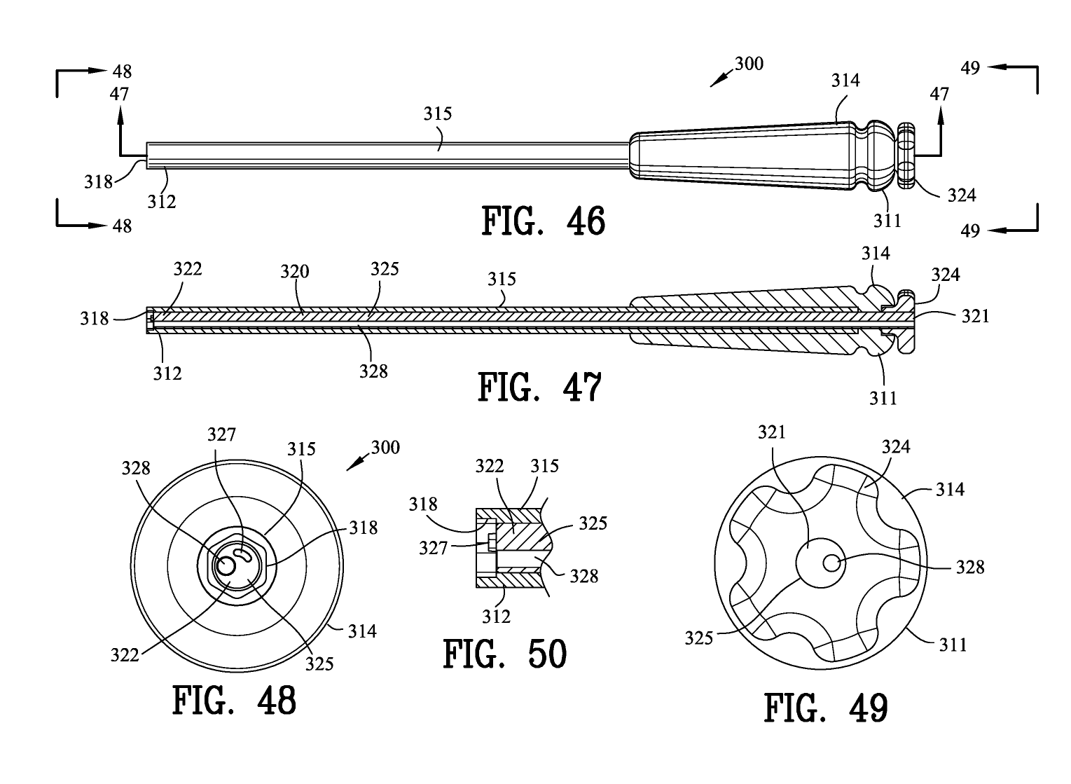

FIGS. 46-50 illustrate a third embodiment of a device 300 suitable for accomplishing the methods set forth in FIGS. 1-13. The third embodiment of a device 300 extends between a proximal end 311 and a distal end 312. The proximal end 311 functions as a handle 314 to rotate the device 300. A shaft 315 is interposed between the proximal end 311 and the distal end 312. The distal end 312 defines a socket 318. In this example, the socket 318 is formed in a shape of a hexagon for receiving the peripheral edge 353 of the suture retention device 240.

A rotator 320 extends between a proximal end 321 and a distal end 322 within the shaft 315 and the handle 314. The proximal end 321 of the rotator 320 functions as a rotator handle 324 to rotate an internal shaft 325 within the shaft 315. The distal end 322 defines an offset protrusion 327 extending therefrom. The internal shaft 325 has a passageway 328 for enabling the flexible member 10 to pass therethrough.

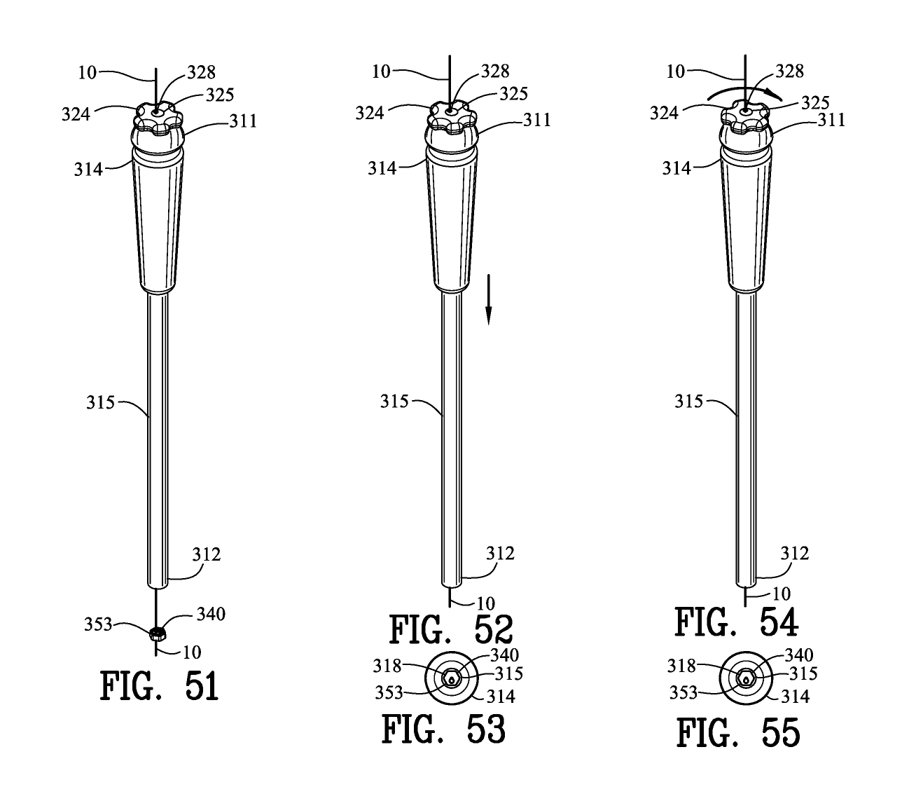

FIG. 51 illustrates the device 300 spaced from the suture retention devices 340 of FIGS. 20-31 with the suture 10 extending through orifices 354 and 364. The suture 10 extends through the passageway 328 of the shaft 325.

FIGS. 52 and 53 illustrate the device 300 of FIG. 50 pushing the suture retention devices 240. The socket 318 at the distal end 312 of the shaft 315 received the peripheral edge 353 of the suture retention device 340. The device 300 moves the suture retention device 340 along the suture 10.

FIGS. 54 and 55 illustrate the device 300 securing the suture retention devices 340 to the suture 10. A rotation of the rotator handle 324 engages the offset protrusion 327 with the hollow 368 defined in the upper surface 362 of the second member 360 of the suture retention devices 340. Continued rotation of the rotator handle 324 rotates the second member 360 relative to the first member 350 for pinching the suture 10 between the first and second members 350 and 360.

The present disclosure includes that contained in the appended claims as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. The method of securing a suture comprising the steps of:
providing a retention device having an orifice;
passing the suture through the orifice in the retention device; and
deforming a portion of the retention device to secure the suture to the retention device.

2. The method of securing a suture comprising the steps of:
providing a retention device having an orifice;
inserting the suture through the orifice in the retention device;
moving the retention device along the suture to position the retention device along; and
deforming a portion of the retention device to secure the suture to the retention device.

3. The method of securing a tissue, comprising the steps of:
providing an anchor having an anchor aperture;
attaching a suture retention device in proximity to a first end of a suture;
passing a second end of the suture through the tissue;
inserting the second end of the suture through the anchor aperture;
pulling the second end of the suture to tighten the suture retention against the tissue; and
securing the second end of the suture.

4. The method of securing a tissue as set forth in claim 3, wherein the step of providing an anchor comprises providing an anchor having a one-way anchor lock; and
the step of securing the suture to the anchor comprises pulling the second end of the suture through the one-way anchor lock for securing the suture in the anchor.

5. The method of securing a tissue as set forth in claim 3, wherein the step of attaching a suture retention device in proximity to a first end of a suture includes the step of crimping the suture retention device to the suture.

6. The method of securing a tissue as set forth in claim 3, wherein the step of attaching a suture retention device in proximity to a first end of a suture includes the step of rotationally securing the suture retention device to the suture.

7. The method of securing a tissue, comprising the steps of
providing an anchor having an anchor aperture;
passing a first end of the suture through a first orifice in a first suture retention device;
securing a first suture fastener to the suture adjacent to the first suture retention device;
passing a second end of the suture through the tissue;
inserting the second. end of the suture into the anchor aperture;
passing the second end of the suture through a second orifice in a second suture retention device;
pulling the second end of the suture to tighten the first suture retention device against the tissue; and
securing a second suture fastener to the suture adjacent to the second suture retention device.

8. The method of securing a tissue as set forth in claim 7, wherein the step of securing a first suture fastener to the suture includes the step of crimping the first suture fastener to the suture.

9. The method of securing a tissue as set thrill in claim 7, wherein the step of securing a first suture fastener to the suture includes the step of rotationally securing the first suture fastener to the suture.

10. The method of securing a first tissue to a second tissue, comprising the steps of:
passing a first end of the suture through a first orifice in a first suture retention device;
securing a first suture fastener to the suture adjacent to the first suture retention device;
passing a second end of the suture through the first tissue;
passing the second end of the suture through the second tissue;
passing the second end of the suture through a second orifice in a second suture retention device;
pulling the second end of the suture to tighten the first suture retention device against the first tissue and the second suture retention device against the second tissue; and
securing a second suture fastener to the suture adjacent to the second suture retention device.

11. The method of securing a tissue as set forth in claim 10, wherein the step of securing a first suture fastener to the suture includes the step of crimping the first suture fastener to the suture.

12. The method of securing a tissue as set forth in claim 10, wherein the step of securing a first suture fastener to the suture includes the step of rotationally securing the first suture fastener to the suture.

13. The method of securing a flexible member, comprising the steps of:
providing a first member having a first orifice;
providing a second member having a second orifice;
locating the second member relative to the first member with the second orifice aligned with the first orifice for enabling the flexible member to extend through the first and second orifices; and
deforming the second member for securing the flexible member.

14. The method of securing a flexible member, comprising the steps of:
providing a first member having a first orifice;
providing a second member having a second orifice;
locating the second member relative to the first member with the second orifice aligned with the first orifice for enabling the flexible member to extend through the first and second orifices; and
rotating the second member relative to the first member for securing the flexible member.

15. A retention device for securing a flexible member, comprising:
a first member having a first orifice;
a second member having a second orifice;
said first member being disposed relative to said second member with said first orifice aligned with said second orifice for enabling the flexible member to extend through said first and second orifices; and
said second member being deformable for securing said flexible member.

16. A retention device for securing a flexible member as set forth in claim 15, wherein said second member is integral with said first member.

17. A retention device for securing a flexible member as set forth in claim 15, wherein said second member is moveable relative to said first member for securing said flexible member.

18. A retention device for securing a flexible member as set forth in claim 15, wherein said second member is moveable relative to said first member for crimping said flexible member.

19. A retention device for securing a flexible member, comprising:
a first member having a first orifice;
a second member having a second orifice;
a rotational mounting for rotatably securing said second member relative to said first member with said tint orifice aligned with said second orifice for enabling the flexible member to extend through said first and second orifices when said first and second members are disposed in a first rotational position; and
said rotational mounting enabling relative rotation of said first and second members into a second rotational position for securing said flexible member.

20. A retention device for securing a flexible member as set forth in claim 19, wherein said second member is rotatable relative to said first member for crimping said flexible member.

21. A retention device for securing a flexible member as set forth in claim 19, including first threads located on said first member; and
second threads located on said second member enabling said second member to threadably engage said first member for crimping to said flexible member.

22. A retention device for securing a flexible member as set forth in claim 19, including first threads located on said first member; and
second threads located on said second member enabling said second member to be thread ably rotated on o said first member for deforming said first member into engagement with said flexible member.

23. A retention device for securing a flexible member, comprising:
a first member having a first orifice;
a second member having a second orifice;
a rotational mounting for rotatably mounting said second member relative to said first member with said first orifice aligned with said second orifice for enabling, the flexible member to extend through said first and second orifices when said first and second members are disposed in a first rotational position; and
a lock for fastening said second member in a second rotational position relative to said first member whereat said first orifice is misaligned with said second orifice for securing said flexible member between said first and second members.

24. A retention device for securing a flexible member as set forth in claim 23, wherein said first member is an outer member and said second member is an inner member; and a retainer for retaining said rotatable inner member relative to said outer member.

25. A retention device for securing a flexible member as set forth in claim 23, wherein said rotational mounting includes a recess defined in said first member for receiving said second member for rotatably mounting said second member relative to said recess.

26. A retention device for securing a flexible member as set forth in claim 23, wherein said lock includes a tooth located on one of said first and second members cooperating with a depression located in said other of said first and second members for maintaining said second member in said second position.

27. A retention device for securing a flexible member as set forth in claim 23, wherein said lock comprises a ratchet interacting between said first and second members for maintaining said second member in said second rotational position.
